(19) United States
(12) Patent Application Publication
(10) Pub. No.: US 2012/0244150 A1
(43) Pub. Date: Sep. 27, 2012

(54) ANTI-VEEV HUMANIZED ANTIBODY

(75) Inventors: Sarah Ann Goodchild, Salisbury (GB); Lyn Margaret O'Brien, Salisbury (GB); Robert John Phillpotts, Salisbury (GB); Stuart David Perkins, Salisbury (GB)

(73) Assignee: The Secretary of State for Defence, Salisbury Wiltshire (GB)

(21) Appl. No.: 13/497,716

(22) PCT Filed: Sep. 20, 2010
(86) PCT No.: PCT/GB2010/001753

§ 371 Date: Mar. 22, 2012

(30) Foreign Application Priority Data
Sep. 22, 2009 (GB) 0916630.7

(51) Int. Cl. A61K 39/42; A61P 31/14; C07K 14/18

(52) U.S. Cl. 424/133.1; 530/387.3

(57) ABSTRACT

The present disclosure relates to an anti-VEEV humanised antibody or a fragment thereof comprising a framework 1, 2, 3, 4, S or 6 CDR regions independently selected from SEQ ID Nos: 2, 3, 4, 5, 6 or 7 characterised in that the antibody or fragment comprises in the framework at least one amino acid, that positively influences the binding/activity of the antibody, from the original murine antibody 1A3B7, pharmaceutical composition comprising same, methods of preparing the antibody or fragment and use of the antibody or fragment in treatment or prophylaxis, in particular the treatment or prophylaxis of VEEV infection.

The present disclosure relates to a humanised anti-VEEV antibody, compositions comprising the same, processes for preparing the antibody and use in the treatment and prophylaxis of Venezuelan equine encephalitis virus.

The Alphavirus Venezuelan equine encephalitis virus (VEEV) is a single stranded, positive-sense RNA virus maintained in nature in a cycle between small rodents and mosquitoes. Six serogroups (I-VI) are currently recognised within the VEEV complex. Spread of epizootic strains of the virus (IA/B and IC) to equines leads to a high viraemia followed by lethal encephalitis and lateral spread to humans. In the human host, VEEV can produce a febrile illness followed in a small proportion of cases by severe encephalitis. Equine epizootics may lead to widespread outbreaks of human encephalitis involving thousands of cases and hundreds of deaths. Viruses in other serogroups do not appear to be equine-virulent and persist in a stable enzootic cycle. Natural transmission of enzootic viruses to humans is rare but may be associated with severe disease.

Epizootic VEEV can be controlled by the immunisation of equines with the attenuated vaccine strain TC-83. TC-83 is solidly protective in equines and has a good safety record. However, in humans it fails to produce protective immunity in up to 40% of recipients and is reactogenic in around 20% of recipients. There have also been reports that the vaccine is potentially diabetogenic and teratogenic. Consequently, TC-83 is no longer available for human use. Both epizootic and enzootic strains of VEEV are infectious for humans by the airborne route and have been responsible for a number of laboratory infections.

In the absence of a suitable vaccine, antiviral therapies that are effective in prophylaxis and treatment of VEEV infection are required. There is evidence to suggest that protection against VEEV requires high antibody levels and, in the case of airborne infection, the presence of antibody on the mucosal surface of the respiratory tract. Previous studies have shown that monoclonal antibodies can protect against VEEV and are effective against disease even when administered 24 h after exposure. Monoclonal antibodies, however, tend to have narrow specificities which limit their use as antiviral therapies. A new broadly reactive antibody which would have the potential to protect against exposure to a range of VEEV strains, is required.

The present disclosure provides humanised antibody with antiviral activity against two or more of serogroups of VEEV.

Thus in one aspect there is provided an anti-VEEV humanised antibody or a fragment thereof comprising a framework and 1, 2, 3, 4, 5 or 6 CDR regions independently selected from SEQ ID Nos: 3, 4, 5, 6, 7 or 8 characterised in that the antibody or fragment comprises in the framework at least one amino acid, that positively influences the binding/activity of the antibody, from the original murine antibody IA3B7.

Each of the variable heavy (V_{H}) and variable light (V_{L}) domains of a traditional antibody molecule are composed of three hyper-variable regions termed Complementary Determining Regions (CDR) separated by more conserved framework regions (FR) (Winter et al., 1994). It is the CDR regions of the antibody that carry the variability in amino acid content and sequence length that give rise to the specificity of any particular antibody molecule. The greatest diversity in length and sequence, thus structural diversity is encoded by the third hyper-variable loop of the heavy chain (V_{H} CDR 3).

Thus CDR as employed herein is intended to refer to a complementary determining region, which is a short amino acid sequence found in the variable domains that complements a particular antigen and provides the antibody or fragment with its specificity for that antigen.

Thus the light chain or fragment thereof will generally contain three CDRs (L1. L2 and L3). The heavy chain or fragment thereof will generally contain three CDRs (H1, H2 and H3). Therefore, when a heavy and a light chain work in co-operation there may be six CDRs that contact that the antigen.

The sequences of the variable domains of the murine antibody 1A3B7 are contained in seq ID No: 1 and 2. The sequence of these variable domains was derived from the messenger RNA taken from the monoclonal cell line producing 1A3B7; an IgG2a isotype antibody with broad VEEV serotype specificity based on specific binding to the E2 viral protein. In in vitro assays this antibody has been shown to neutralise infective virus when tested with vero cell plaque assays. This neutralising activity is thought to play a significant role in the protective effects of this antibody, which has been shown to be protective against disease induced by exposure to mouse virulent strains of VEEV from the serotypes I, II and IIIA through the aerosol route.

The direct treatment of humans with antibodies from mice has found some limited utility, e.g. mouse monoclonal antibody, orthoclone OKT3, has been used to prevent organ rejection. The direct use of animal antibodies can however be limited by two problems. Firstly, antibodies from different animal species may not interact properly with Fc receptors and/or complement leading to a lack of appropriate down-stream effector functions. Secondly, antibodies from non-human species are recognised as “foreign” by the human immune system. Repeated administration of such antibodies can therefore result in an immunogenic response sometimes referred to as the human anti-mouse antibody response (HAMA). The generation of such a response can severely limit the application of antibodies by reducing the therapeutic window through a rapid clearance of antibody from the system and the possibility of a severe immunogenic response that could include anaphylactic shock, cytokine storm and the like.

Problems associated with inconsistent effector function of murine monoclonals have been alleviated through the generation of “chimeric” antibodies where the variable domains, variable heavy (V_{H}) and variable light (V_{L}), of a murine antibody are grafted onto the constant domains of a human antibody molecule. This approach also removes the majority of the immunogenic portion of the mouse antibody molecule replacing it with human protein. This approach does not however, always provide a reproducible means of fully reducing immunogenic responses to the chimeric antibodies to acceptable levels in vivo. Consequentially a number of protein engineering methods have been developed to reduce the murine, or other non-human, content of antibody molecules to a minimal level. These approaches are collectively termed “humanisation”.

Based on this information, one method by which the humanisation of antibodies can be undertaken is to take the amino acid sequences of the CDR regions of a candidate murine antibody and insert them into the FR regions of a human antibody. This reduces the murine content of the antibody molecule to the CDR regions only. Humanised antibody or fragment as employed herein is intended to refer to where one or more of the CDRs is/are from a non-human species such as mouse and the framework/immunoglobulin structure is human or substantially human.

The human framework employed to support the grafted CDR regions may, for example be performed by searching databases such as blast searches to identify human variable heavy and/or variable light chain sequences similar to those in the murine antibody. The CDR(s) from the murine antibody can then be grafted onto this framework, as appropriate. These frameworks can also be grafted onto the human heavy chain constant regions and light chain constant regions to assemble a whole humanised antibody. Different antibody isotypes can be generated by grafting the humanised variable domains onto the relevant constant domains i.e. IgM, IgG, IgA and IgD and sub-types thereof, namely G1, G2, G3, G4, A1 and A2.

As a consequence of undertaking the humanisation process, it is possible to affect the biophysical and biological integrity of a humanised antibody in comparison to the parent molecule. A failure to retain the properties of the molecule through the process of humanisation may lead to limitations in use, rendering candidate antibodies inappropriate for use as a therapeutic agent. Key amino acid residues or framework structure necessary to retain antibody function are not predictable and many examples exist in the literature describing loss of specificity, reduction in affinity and biophysical integrity in comparison to the non-human antibody. As a consequence when humanisation of antibodies is undertaken it is usually necessary to produce several variants of the humanised molecule and then select from this panel the molecule that best retains the biological activity of the parent antibody. In addition, it may be necessary to refine the characteristics of the humanised antibody through mutation and maturation to reinstate or optimise desirable properties of the molecule.

In the case of humanisation of the VEEV specific antibody 1A3B7, retention of at least one specific amino acid residue from the murine framework has been shown to be important in the retention of activity. When at least one of the murine framework residues is not present in the humanised antibody there is a significant loss of activity.

Significant loss of activity as employed herein is intended to refer to a 50% or more loss of specificity of the antibody, for example to the E2 viral protein, a 50% loss in neutralisation activity in the vero cell plaque assay referred to herein and/or loss of protective properties in vivo against viral challenge. The effect of any amino acid substitutions, additions and/or deletions can be readily tested by one skilled in the art, for example by using the in vitro assays, for example a BIAcore assay and/or said vero cell plaque assay.

In one embodiment the least one amino acid from the original murine antibody 1A3B7 is 1, 2, 3, 4 or 5 amino acid residues therefrom. The residues may be in the heavy chain framework only or the heavy and light chain framework.

In one embodiment the at least one amino acid from the original murine antibody 1A3B7 is located in the heavy chain framework.

In one embodiment no amino acid residues from the murine framework are retained in the light chain.

Retention of amino acids from the murine framework as employed herein is intended to refer to modification/mutation of the human framework to ensure that an amino acid located in the murine framework is located in a corresponding position in the human framework.

In one embodiment the at least one amino acid from the original murine antibody 1A3B7 is an isoleucine residue, for example corresponding to isoleucine H94 (Kabat numbering) in FR3 (framework region 3) in the original murine antibody 1A3B7.

In an alternative aspect the present disclosure provides antibody or fragment wherein the isoleucine corresponding to isoleucine H94 (Kabat numbering) in FR3 in the original murine antibody 1A3B7 is conservatively substituted by a residue, for example leucine or valine.

Thus in one embodiment the human framework employed comprises an isoleucine amino acid corresponding to isoleucine H94 (Kabat numbering) in FR3 in the original murine antibody 1A3B7.

In one embodiment the antibody or fragment according to the present disclosure comprises at least the CDR sequence of Seq ID No: 5 and an isoleucine amino acid corresponding to isoleucine H94 (Kabat numbering) in framework 3 region (FR3) in the original murine antibody 1A3B7.

Retention of CDR3 (seq ID No: 5) and an isoleucine amino acid in the position which corresponds to isoleucine 1-194 in the heavy chain of the murine antibody results in good retention of the affinity of the humanised antibody in comparison to the murine parental molecule. In addition, the neutralising activity and broad specificity of the molecule is comparable to the murine counterpart. Consequentially, the function of the molecule is sufficient to represent a useful therapeutic candidate for VEEV.

Positive in the context of the present disclosure is intended to refer to the presence of the amino acid residue in the antibody or fragment has a beneficial effect to one or more properties of the modified antibody in comparison to the absence of the amino acid residue.

Neutralising in the context of the present disclosure is intended to refer to wherein the antibody reduces or abolishes some biological activity, such as the ability of the virus to infect cells (such as in the vero cell plaque assay), for example a reduction of 10, 20, 30, 40, 50, 60, 70, 80, 90 or 100%.

Thus the humanised antibody according to the disclosure is potentially useful as a therapeutic agent against VEEV because it retains the broad spectrum of activity, the neutralising characteristics and/or the good level of activity of the murine counterpart.

As employed herein isoleucine in the humanised antibody corresponding to isoleucine H94 in the murine antibody is intended to refer to the fact that the humanised antibody has an isoleucine amino acid in a position that correlates with the isoleucine H94 found in the murine antibody. Thus, for example when the two sequences are aligned there is substantial similarity in the relevant section and isoleucine is found in the humanised sequence in a similar or identical position to the position of isoleucine in the murine antibody, even if there is not exact identity with the absolute amino acid numbers assigned in each sequence.

Sequence alignments and comparisons may be performed, for example employing BLAST analysis, or similar suitable software. Degrees of identity and similarity can be readily calculated using known computer programs. For example, simple sequence comparisons can be done on web-sites such as the NCBI website: http://www.ncbi.nlm.nih.gov/BLAST/ (version 2.2.11). As used herein, percentages identity or similarities between sequences are measured according to the default BLAST parameters, version 2.2.11.

“Identity”, when referring to a polypeptide, indicates that at any particular position in the aligned sequences, the amino acid residue is identical between the sequences. “Similarity”, when referring to a polypeptide, indicates that, at any particular position in the aligned sequences, the amino acid residue is of a similar type between the sequences. Amino acid residues can be grouped by their side chains. Amino acids within a specific group are regarded as of a similar type. Glycine, alanine, valine, leucine and isoleucine all have aliphatic side-chains and amino acids in this group may be regarded as similar. Proline, although a cyclic amino acid, shares many properties with the aliphatic amino acids and may also be regarded as being grouped with the other aliphatic amino acids. Another group is the hydroxyl or sulphur containing side chain amino acids. These are serine, cysteine, threonine and methionine.

Phenylalanine, tyrosine and tryptophan are grouped together as the aromatic amino acids. Histidine, lysine and arginine are in the group of basic amino acids. Aspartic acid and glutamic acid are in the group of acidic amino acids, and asparagine and glutamine are in the group of their respective amides. Also included in the groups are modified amino acids (i.e. non-naturally occurring amino acids) that have side-chains that share similar properties with the naturally occurring amino acids. Members of a particular group can be regarded as being “similar”. Swapping one amino acid from a group with another amino acid from the same group is often termed a conservative substitution.

In one aspect the antibody or fragment thereof according to the disclosure comprises a light chain variable region sequence of Seq ID No: 12 or a sequence 90% similar or identical thereto.

In one aspect the antibody or fragment thereof according to the disclosure comprises a heavy chain variable region sequence of Seq ID No: 11 or a sequence 90% similar or identical thereto.

In one aspect the antibody or fragment thereof according to the disclosure comprises a light chain variable region sequence of Seq ID No: 12 or a sequence 90% similar or identical thereto and a heavy chain variable region sequence of Seq ID No: 11 or a sequence 90% similar or identical thereto.

In one embodiment the light chain framework of Seq ED No: 9 or a derivative thereof is employed in the antibody or fragment of the disclosure.

In one embodiment a heavy chain framework of Seq ID No: 10 or a derivative thereof is employed in the antibody or fragment of the disclosure.

Derivative as employed in the context of frameworks is intended to refer to where modifications are made to the original framework but the construct formed still retains it essential characteristics, for example retaining 90% sequence identity over the length of the whole framework.

Fragments of antibodies include domain antibodies (i.e. a single variable region characterised in that they contain a murine amino acid in the framework), for example from the heavy or light chain variable region, single chains such as the heavy chain or light chain, Fab fragments which comprise the variable region of a light and heavy chain or a Fab′ fragments which comprise the variable region of a light and heavy chain and a small portion of the constant region of each chain, up to and including the hinge region. In one embodiment the fragment is a F(ab′)₂ or a single chain Fv fragment (wherein a V_{H} and V_{L} are joined). Alternatively the fragment may be a full length heavy chain and a full length light chain pairing. In one embodiment the antibody or fragment is comprised in a multivalent or bispecific molecule. The disclosure also extends to conjugates of the fragments described herein.

Particular examples of antibody fragments for use in the present disclosure are Fab′ fragments which possess a native or a modified hinge region. A number of modified hinge regions have already been described, for example, in U.S. Pat. No. 5,677,425, WO 99/15549, and WO 98/25971 and these are incorporated herein by reference.

In one embodiment the fragment is a functionally binding fragment.

There are different types of antibodies, which may be employed in the disclosure such as IgM, IgG, IgA and IgD and sub-types thereof, namely G1, G2, G3, G4, A1 and A2. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. Sequence variants of these constant region domains may also be used. For example IgG4 molecules in which the serine at position 241 has been changed to praline as described in Angal et al., Molecular Immunology, 1993, 30 (1), 105-108 may be used.

In one embodiment the antibody or fragment comprises an Fc region, for example with an effector function.

In one embodiment the antibody or fragment comprises an Fc region without an effector function.

In one embodiment the antibody or fragment thereof is IgG, for example IgG2, such as IgG2a.

In one embodiment of the disclosure a heavy chain is a mu, gamma, delta or epsilon isotope.

In one embodiment of the disclosure a light chain is a kappa or lambda isotope, such as kappa, in particular kappa B1. Kappa B1 advantageously is able to accommodate the long CDR L1 and may ultimately have a beneficial effect on affinity. Alternatively, simply the framework region from Kappa B1 may be employed, as appropriate.

En one embodiment there is provided a complete antibody comprising at least 6 CDRs in two variable domains and heavy and light constant regions. The antibody may optionally comprise further variable domains to the same or a different antigen.

In the example of this humanised version of 1A3B7 the combination of the human germline light chain B1 and human germline heavy chain DP-75 ensures retention of the broad specificity and affinity of binding of the parent murine antibody. In addition, it is essential to retain a non-typical isoleucine amino acid residue within the framework 3 region of the heavy chain adjacent to the CDR3 region (H94, kabat numbering scheme).

The methods for creating these antibody molecules are well known in the art. The types of expression systems available to produce these antibody molecules include bacterial, yeast, insect and mammalian expression systems, the methods for which are well known in the art.

It will be appreciated that one or more amino acid substitutions, additions and/or deletions may be made to the antibody variable domains, provided by the present invention, without significantly altering the advantageous properties of the antibody or fragment.

Antibodies may undergo a variety of posttranslational modifications. The type and extent of these modifications often depends on the host cell line used to express the antibody as well as the culture conditions. Such modifications may include variations in glycosylation, and deamidation.

Any of the embodiments defined herein may comprise a CDR, nominally referred to herein as H1, for example with the sequence shown in Seq ID No: 3 (or this sequence wherein one amino acid has been replaced).

Any of the embodiments defined herein may comprise a CDR, nominally referred to herein as H2, for example with the sequence shown in Seq ID No: 4 (or this sequence wherein one amino acid has been replaced).

Any of the embodiments defined herein may comprise a CDR, nominally referred to herein as H3 for example with the sequence shown in Seq ID No: 5 (or this sequence wherein one amino acid has been replaced).

Any of the embodiments defined herein may comprise a CDR, nominally referred to herein as L1, for example with the sequence shown in Seq ID No: 6 (or this sequence wherein one amino acid has been replaced).

Any of the embodiments defined herein may comprise a CDR, nominally referred to herein as L2, for example with the sequence shown in Seq ID No: 7 (or this sequence wherein one amino acid has been replaced).

Any of the embodiments defined herein may comprise a CDR, nominally referred to herein as L3, for example with the sequence shown in Seq ID No: 7 (or this sequence wherein one amino acid has been replaced).

The disclosure also extends to embodiments comprising the following combination of CDRs:

H1 and L1, H1 and L2, H1 and L3, H1 and H2, H1 and H3, H2 and L1, H2 and L2, H2 and L3, H3 and L1, H3 and L2, H3 and L3, L1 and L2, L1 and L3, H1 and H2 and L1, H1 and H2 and L2, H1 and H2 and L3, H1 and H2 and H3, H1 and H3 and L1, H1 and H3 and L2, H1 and H3 and L3, H2 and H3 and L1, H2 and H3 and L2, H2 and H3 and L3, H1 and H2 and H3, L1 and L2 and H1, L1 and L2 and H2, L1 and L2 and H3, L1 and L2 and L3, H1 and H2 and H3 and L1, H1 and H2 and H3 and L2, H1 and H2 and H3 and L3, L1 and L2 and L3 and H1, L1 and L2 and L3 and H2, L1 and L2 and L3 and H3, H1 and H2 and H3 and L1 and L2, H1 and H2 and H3 and L1 and L3, H1 and H2 and H3 and L2 and L3, L1 and L2 and L3 and H1 and H2, L1 and L2 and L3 and H1 and H3, L1 and L2 and L3 and H2 and H3, or H1 and H2 and H3 and L1 and L2 and L3, as defined herein. In this embodiment H1, H2, H3, L1, L2 and L3 refers to the nomenclature in the sequence listing herein and may also refer to the position in the variable region in the antibody or fragment formed.

In one embodiment CDR1 in the murine antibody 1A3B7 is CDR1 in the humanized antibody according to the disclosure.

In one embodiment CDR2 in the murine antibody 1A3B7 is CDR2 in the humanized antibody according to the disclosure.

In one embodiment CDR3 in the murine antibody 1A3B7 is CDR3 in the humanized antibody according to the disclosure.

In one embodiment CDR4 in the murine antibody 1A3B7 is CDR4 in the humanized antibody according to the disclosure.

In one embodiment CDR5 in the murine antibody 1A3B7 is CDR5 in the humanized antibody according to the disclosure.

In one embodiment CDR6 in the murine antibody 1A3B7 is CDR6 in the humanized antibody according to the disclosure.

In one embodiment the antibody or fragment according to the disclosure comprises 6 CDRs selected from sequence 3 to 8.

The disclosure also extends to sequences with 80%, such as 85%, 90%, 95%, 96%, 97%, 98% or 99% sequence identity to a sequence herein, for example when the comparison is performed against the full sequence disclosed or a relevant portion of a larger sequence, for example signal sequences used to target production of antibody to sub-cellular, extracellular environments in an appropriate heterologous expression system.

Functionally binding fragment as used herein refers to a fragment that recognises/binds the same entities or substantially the same entities as the corresponding full antibody, although not necessarily with the same affinity or avidity, but nonetheless can be used to perform a corresponding function to that of the full antibody.

Suitably antibodies and fragments of the disclosure are specific for one or more VEEV epitopes.

Specific in the context of the present disclosure is intended to mean that the antibody or fragment primarily recognises and interacts with a VEEV epitope and has a higher affinity and/or avidity for that epitope than is does for any other entity.

In one embodiment a fragment or an antibody of the disclosure provides is linked to a biological reporter system such as an enzyme by means such as chemical cross-linking or genetic manipulation.

Antibodies, fragments and/or derivative according to the present disclosure may be administered in combination with an effector molecule, for example the effector molecule may increase half-life in vivo, and/or decrease immunogenicity and/or enhance the delivery of an antibody across an epithelial barrier to the immune system. Examples of suitable effector molecules include polymers and proteins such as albumin and albumin binding proteins. Examples of suitable polymers include any synthetic or naturally occurring substantially water-soluble, substantially non-antigenic polymer including, for example, optionally substituted straight or branched chain polyalkylene, polyalkenylene, or polyoxyalkylene polymers or branched or unbranched polysaccharides, e.g. a homo- or hetero-polysaccharide such as lactose, amylose, dextran or glycogen. Particular optional substituents which may be present on the above-mentioned synthetic polymers include one or more hydroxy, methyl or methoxy groups. Particular examples of synthetic polymers include optionally substituted straight or branched chain poly(ethyleneglycol), poly(propyleneglycol), poly(vinylalcohol) or derivatives thereof, especially optionally substituted poly(ethyleneglycol) such as methoxypoly(ethyleneglycol).

In one embodiment the polymer is a polyalkylene oxide such as polyethylene glycol (PEG).

In one example antibodies or fragments of the present disclosure are attached to poly (ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods. See for example U.S. Pat. No. 5,219,996. Multiple sites can be used to attach two or more PEG molecules. Suitably PEG molecules are covalently linked through a thiol group of at least one cysteine residue located in the antibody fragment. Where a thiol group is used as the point of attachment appropriate agents, for example thiol selective derivatives such as maleimides and cysteine derivatives may be used to effect the coupling.

The antibody may, for example a modified Fab fragment, such as a Fab′ which is PEGylated, i.e. has PEG (poly (ethyleneglycol)) covalently attached thereto, e.g. according to the method disclosed in EP 0948544. The total amount of PEG attached to the fragment may be varied as desired, but will generally be in an average molecular weight range from 250 to 100,000 Da, for example from 5,000 to 50,000 Da, such as from 10,000 to 40,000 Da and particularly from 20,000 to 40,000 Da. The size of PEG may, in particular, be selected on the basis of the intended use of the product, for example ability to local in to certain tissues or extend circulating half-life.

The reduction and PEGylation reactions may generally be performed in a solvent, for example an aqueous buffer solution such as acetate or phosphate, at around neutral pH. for example around pH 4.5 to around pH 8.5, typically pH 4.5 to 8, suitably pH 6 to 7. The reactions may generally be performed at any suitable temperature, for example between about 5° C. and about 70° C., for example at room temperature. The solvent may optionally contain a chelating agent such as EDTA, EGTA, CDTA or DTPA. Suitably the solvent contains EDTA at between 1 and 5 mM, such as 2 mM. Alternatively or in addition the solvent may be a chelating buffer such as citric acid, oxalic acid, folic acid, bicine, tricine, tris or ADA. The PEG will generally be employed in excess concentration relative to the concentration of the antibody fragment. Typically the PEG is in between 2 and 100 fold molar excess, for example 5, 10 or 50 fold excess.

Where necessary, the desired product containing the desired number of PEG molecules may be separated from any starting materials or other product generated during the production process by conventional means, for example by chromatography techniques such as ion exchange, size exclusion, protein A, G or L affinity chromatography or hydrophobic interaction chromatography.

The disclosure provides an antibody or fragment thereof that is at least bispecific, that is to say that they recognise at least two strains of the VEEV, such as three, four or five strains of VEEV, in particular all known strains of VEEV capable of causing an epidemic in animals (eg subtypes IA/B and IC), especially viruses from subtypes IA/B, IC, ID, IE, IF, II, IIIA, IV, V and VI or all known strains of VEEV.

In one aspect there is provided a pharmaceutical composition comprising an antibody or fragment as defined herein.

Pharmaceutical compositions may be conveniently presented in unit dose forms containing a predetermined amount of an active agent of the disclosure per dose.

Pharmaceutically acceptable carriers may take a wide variety of forms depending, e.g. on the route of administration.

Typical delivery routes include parenteral administration, e.g., intradermal, intramuscular or subcutaneous delivery. Other routes include oral administration, intranasal, intravaginal routes, intradermal and transdermal administration.

In one embodiment the antibody or fragment according to the disclosure is provided optionally as a lyophilized formulation for reconstitution later or as a liquid formulation for infusion or injection.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents as known in the art. Having said this, precautions will usually be required to protect the antibody or fragment from degradation by stomach acid. Alternatively liquid or solid formulations may be administered sublingually or through a buccal membrane.

In the case of oral solid preparations such as powders, capsules and tablets, carriers such as starches, sugars, microcrystalline cellulose, granulating agents, lubricants, binders, disintegrating agents, and the like may be included. In addition to the common dosage forms set out above, active agents of the invention may also be administered by controlled release means and/or delivery devices. Tablets and capsules may comprise conventional carriers or excipients such as binding agents for example, syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated by standard aqueous or non-aqueous techniques according to methods well known in normal pharmaceutical practice. An enteric coating may be employed to protect the antibody or fragment from degradation in the stomach or intestines.

Pharmaceutical compositions suitable for oral administration may be presented as discrete units such as capsules, cachets or tablets, each containing a predetermined amount of the active agent, as a powder or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active agent with the carrier, which constitutes one or more necessary ingredients. In general, the compositions are prepared by uniformly and intimately admixing the active agent with liquid carriers or finely divided solid carriers or both, and then, if necessary, shaping the product into the desired presentation. For example, a tablet may be prepared by compression or moulding, optionally with one or more accessory ingredients.

Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils. These preparations generally contain a preservative to prevent the growth of microorganisms.

The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. Extemporaneous injection solutions, dispersions and suspensions may be prepared from sterile powders, granules and tablets.

The active agents can be incorporated, if desired, into liposomes, microspheres or other polymer matrices Liposomes, for example, which consist of phospholipids or other lipids, are nontoxic, physiologically acceptable and metabolizable carriers that are relatively simple to make and administer.

Liposome carriers may serve to target a particular tissue or infected cells, as well as increase the half-life of the antibody or fragment. Liposomes include emulsions, foams, micelles, insoluble monolayers, liquid crystals, phospholipid dispersions, lamellar layers and the like. In these preparations the vaccine to be delivered is incorporated as part of a liposome, alone or in conjunction with a molecule which binds to, e.g., a receptor prevalent among lymphoid cells, such as monoclonal antibodies or with other therapeutic or immunogenic compositions. Thus, liposomes either filled or decorated with a desired immunogen of the disclosure can be directed to the site of lymphoid cells, where the liposomes then deliver the immunogen(s). Liposomes may be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., U.S. Pat. Nos. 4,235,871, 4,501,728, 4,837,028, and 5,019,369.

The liposomes generally contain a neutral lipid, for example phosphatidylcholine, which is usually non-crystalline at room temperature, for example egg yolk phosphatidylcholine, dioleoyl phosphatidylcholine or dilauryl phosphatidylcholine.

In one embodiment the disclosure provides a pharmaceutical composition for infusion.

In one embodiment the formulation/composition is a vaccine.

Vaccine preparation techniques are generally well known. Encapsulation within liposomes is described, for example in U.S. Pat. No. 4,235,877.

In one embodiment the formulation is provided as a formulation for topical administrations including inhalation.

Suitable inhalable preparations include inhalable powders, metering aerosols containing propellant gases or inhalable solutions free from propellant gases. Inhalable powders according to the disclosure containing the active agent may consist solely of the above-mentioned active agents or of a mixture of the above-mentioned active agents with physiologically acceptable excipient.

These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are preferably used, the use of lactose or glucose, particularly but not exclusively in the form of their hydrates.

Particles for deposition in the lung require a particle size less than 10 microns, such as 1-9 microns for example from 0.1 to 5 μm, in particular from 1 to 5 μm.

The propellent gases which can be used to prepare the inhalable aerosols are known in the art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof.

Particularly suitable propellent gases are halogenated alkane derivatives selected from among TG 11, TG 12, TG 134a and TG227. Of the abovementioned halogenated hydrocarbons, TG 134a (1,1,1,2-tetrafluoroethane) and TG227 (1,1,1,2,3,3,3-heptafluoropropane) and mixtures thereof are preferred according to the invention.

The propellent-gas-containing inhalable aerosols may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active agent. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active agent.

In one embodiment the dose is in the range 1 pg to 100 mg per Kg, such as 1 ng to 10 mg per Kg.

Pharmaceutical compositions can be administered with medical devices known in the art. For example, in one embodiment, a pharmaceutical composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollients in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the active agent in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are suitably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

Pharmaceutical compositions adapted for topical administration in the mouth include lozenges, pastilles and mouth washes.

Pharmaceutical compositions suitable for rectal administration wherein the carrier is a solid are most suitably presented as unit dose suppositories. Suitable carriers include cocoa butter or other glyceride or materials commonly used in the art, and the suppositories may be conveniently formed by admixture of the combination with the softened or melted carrier (s) followed by chilling and shaping moulds. They may also be administered as enemas.

The dosage to be administered will vary according to the subject, and the nature and severity of the infection and the physical condition of the subject, and the selected route of administration; the appropriate dosage can be readily determined by a person skilled in the art.

The compositions may contain from 0.1% by weight, for example from 10-60%, or more, by weight, of the active agent, depending on the method of administration.

It will be recognized by one of skill in the art that the optimal quantity and spacing of individual dosages of an antibody or fragment of the disclosure will be determined by the nature and extent of the condition being treated, the form, route and site of administration, and the age and condition of the particular subject being treated, and that a physician will ultimately determine appropriate dosages to be used. This dosage may be repeated as often as appropriate.

If side effects develop the amount and/or frequency of the dosage can be altered or reduced, in accordance with normal clinical practice.

In one embodiment there is provided a solution or suspension of an antibody, fragment or derivative according to the disclosure, for example in an organic or aqueous solvent.

In one embodiment the antibody, fragment or derived according to the disclosure is lyophilized or frozen.

In one aspect there is provided an antibody, fragment or pharmaceutical composition as defined herein for use in treatment, in particular for use in the prophylaxis and/or treatment of VEEV infection.

In one aspect there is provided an antibody, fragment or pharmaceutical composition as defined herein for use in the manufacture of a medicament for the treatment or prophylaxis of VEEV infection.

In one aspect there is provided a method of treatment comprising administering a therapeutically effective amount of an antibody, fragment or pharmaceutical composition as defined herein, in particular for the prophylaxis or treatment of VEEV infection.

In one embodiment the antibody, fragment or pharmaceutical compositions comprising same is administered before exposure to the virus.

In one embodiment the antibody, fragment or pharmaceutical compositions comprising same is administered up to 24 hours after exposure to the virus.

In one embodiment the antibody, fragment or pharmaceutical compositions comprising same is administered before exposure to the virus and up to 24 hours after exposure to the virus.

In one embodiment there is provided a polynucleotide, for example DNA encoding an antibody or fragment defined herein.

In one embodiment there is provided a vector comprising a polynucleotide, for example DNA encoding an antibody or fragment defined herein.

In one embodiment there is provided a host comprising a polynucleotide, for example DNA encoding an antibody or fragment defined herein.

Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

The present invention also provides a process for the production of an antibody or fragment according to the present invention comprising culturing a host cell containing a vector (and/or DNA) of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

In one aspect there is provided a method of humanising a murine antibody 1A3B7 comprising grafting at least CDR of seq ID No: 5 into an appropriate framework and retaining an isoleucine amino acid corresponding to isoleucine H94 in the original murine antibody 1A3B7.

It is also envisaged that one or more embodiments described herein may be combined, as technically appropriate.

In the context of this specification “comprising” is to be interpreted as “including”.

Aspects of the disclosure comprising certain elements are also intended to extend to alternative embodiments “consisting” or “consisting essentially” of the relevant elements.

### EXAMPLES

### Materials and Methods

### Cells and Viruses

The L929 (murine fibroblast), HEK 293 (human kidney) and Vero (simian kidney) cell lines (European Collection of Animal Cell Cultures, U.K.) were propagated by standard methods using the recommended culture media. Stocks of VEEV vaccine strain TC-83 were propagated from a vial of vaccine originally prepared for human use (National Drug Company, Philadelphia, U.S.A.). Strains of VEEV from serogroups IA/B (Trinidad donkey: TrD). IC (P676), ID (3880), IE (Mena II), IF (78V), II (Fe37c), IIIA (BeAn8), IV (Pixuna), V (CaAr508) and VI (AG80) were kindly supplied by Dr. B. Shope (Yale Arbovirus Research Unit, University of Texas. U.S.A.). Virulent virus stocks were prepared and the titre determined as described by Phillpotts R. (2006) Virus Research, 120, 107-112. All work with virulent VEEV was carried out under U.K. Advisory Committee on Dangerous Pathogens Level 3 containment.

### Harvesting of Genes Encoding the Variable Heavy and Variable Light Chain Domains of Anti-VEEV Antibody 1A3B7

The Hybridoma cell line 1A3B7 was revived from storage in liquid nitrogen and grown in Dulbecco's modification of Eagle's medium (Gibco BRL) supplemented with 10% foetal calf serum (DF10) plus pen/strep (Gibco BRL) and 100 μM sodium pyruvate. Samples of the media containing secreted antibody from this cell line were analysed using a murine monoclonal isotype analysis kit (Amersham). This confirmed that the cell line produced a murine immunoglobulin of IgG2a/kappa isotype. Log phase cells were harvested and used to prepare RNA using an RNeasy midi-prep kit (Qiagen). The concentration and quality of the RNA was measured by spectrophotometry using a Gene Quant RNA/DNA calculator (Pharmacia). The RNA (50-500 ng) was then used to generate cDNA using a superscript RT-PCR kit (Invitrogen). DNA fragments encoding the variable light and variable heavy chains of the 1A3B7 antibody were rescued from the cDNA using PCR with primer pools specific for the variable domains of each antibody domain. The resultant amplicons were then cloned into pGEM T vector (Promega) and analyzed by DNA sequencing.

Generation scAb Molecule from 1A3B7 V_{H} and V_{L} and Analysis of Antigen Binding Activity

The sequences encoding the variable domains for antibody 1A3B7 were used to design specific oligonucleotides to facilitate the construction of a linked single chain variable fragment (scFv) segment encoding the variable regions with a Sfi I site at the 5′ terminus and a Not I site at the 3′ terminus. This scFv was then cloned into the expression vector pHAP Express (Haptogen) to facilitate periplasmic production of recombinant scFv carrying a human kappa domain as a fusion protein (termed 1A3B7 scAb). The recombinant 1A3B7 scAb was dialysed against PBS and quantified using a Bradford Assay prior to use in activity assays. Recombinant 1A3B7 scAb protein was then assessed by ELISA for binding to inactivated VEEV TC83 (1 μg/ml) using an human C-kappa light chain specific detection antibody antibody HRP (Sigma) diluted 1/1000 in PBS to confirm that the V_{H} and V_{L} domains combined to provide antigen binding as expected.

### Analysis of Antibody Sequences and Identification of Candidate Germline Sequences for Humanisation of Antibody 1A3B7

DNA sequences encoding antibody gene fragments were analysed using either DNA for windows software or DNAStar™ both of which allow for analysis of sequence for each of the 3 codon reading frames and in both directions. Assignment of kabat numbering to the V_{L} and V_{H} chains of 1A3B7 was performed using Andrew Martin's Kabat sequence analysis tools (http://www.bioinforo.uk/abs/simkab.html). Alignment of the sequences for the V_{H} and V_{L} to potential human germline candidates for humanisation was performed using NCBI IgB LAST tools (http://www.ncbi.nlm.nih.gov/igblast/)

### Production and Purification of Recombinant Chimeric and Humanised 1A3B7 in Mammalian Cell Culture

The 1A3B7 chimeric antibody was constructed using the murine V_{L} and V_{H} domains harvested from the 1A3B7 hybridoma cell line. These variable domains were fused to human IgG1 or κ constant regions and then cloned as Hind III/Mfe I fragments into the eukaryotic expression vector pCMVScript (Strategene). Host cell lines, Chinese hamster ovary (European Collection of Animal Cell Cultures, Porton) (CHO DG44) were co-transfected with both the heavy and light chain containing vector DNAs and grown in selective medium after selection with Geneticin (Invitrogen). Transfected cells were plated out in 96 well plates at a density of ½ cell per well (200 μl medium per well).

Humanised versions of the V_{H} and V_{L} regions of 1A3B7 were synthetically generated and amplified using PCR to add compatible restriction enzyme sites at the 5′ and 3′ ends to facilitate cloning into antibody expression vector (pHEE, Haptogen). In the case of the V_{H} genes the restriction sites used were Eco RI/Sca I and the kappa light chains have Bam HI/Bsi WI sites. Use of these sites allows the insertion of these humanised genes into expression vectors in frame with human kappa and IgG1 constant regions. Three humanised V_{H} genes and three humanised V_{L} genes were designed. These variants were cloned in all nine possible combinations into the pHEE expression system. A DNA sample from each of these nine constructs was sent for sequencing and determined to be correct. Complete vectors harbouring the humanised 1A3B7 antibody constructs were transfected into CHO DG44 cells using Lipofectamine 2000 (Invitrogen) in accordance with the manufactures instructions. Transient expression was assessed after 60 hours. The quantity of antibody produced by the recombinant cell lines was assessed by capture ELISA

To produce significant quantities of purified chimeric or humanised antibody, CHO DG44 cell lines that showed resistance to Geneticin (Invitrogen) were propagated in IMDM (Gibco BRL) supplemented with 10% FBS, antimycotic, Gentamycin, sodium pyruvate, pen/strep, glutamine, NEAA and AA and methotrexate (10 nM) using gentecin (400 μg/ml) selection to isolate transformed cells (all additives were from Gibco BRL unless otherwise stated). Cell lines secreting antibody were expanded and the highest producers selected. Humanised antibody was purified via protein A affinity chromatography using Prosep®-A (Bioprocessing Ltd). The antibody was then dialysed into PBS and quantified by capture ELISA followed by analysis on denaturing SDS-PAGE gels to confirm the presence of the heavy and light chains of the antibody molecule prior to use in in vitro activity assays.

### Capture ELISA to Determine Monoclonal Antibody Concentration

Antibody was captured onto an immulon 4 ELISA plate using goat ant-mouse IgG (Whole molecule, Sigma) for murine antibodies, anti-human (Sigma) to capture chimeric and humanised forms of 1A3B7 and goat anti-human kappa light chain (Sigma) to capture scAb forms of IA3137 carrying a human κ domain. Samples of antibody for analysis were then added to each well of the ELISA plate and double diluted across the plate. Samples of standard antibodies of known concentrations were added as positive controls and to allow for quantification of the antibody. Secondary anti-species (mouse or human) detection antibodies conjugated to Horseradish peroxidise were then added to detect the bound antibody.

### Testing of the Activity of Murine Monoclonal and Recombinant Forms of Anti-VEEV IgG 1A3B7 In Vitro

The ability of antibodies to recognise a variety of VEEV strains was tested by ELISA using sucrose density gradient-purified antigen from strains TrD, P676, 3880, Mena II, 78V, Fe37c, BeAn8, Pixuna, CaAr508 and AG80. So that the reactivity could be meaningfully compared, the VEEV antigens used in the ELISA were first examined by SDS-PAGE and scanning densitometry. Each antigen was diluted in coating buffer to contain an equivalent amount of virus glycoprotein. The ability of the antibody to neutralise virus infectivity was also determined. Appropriate amounts of antibody was mixed with VEEV strains TrD. Fc37c or BeAn8 (approximately 100 pfu) and incubated at 4° C. overnight. Residual infectious virus was estimated by plaque assay in L929 cells.

In Vivo Protection of Mice with Humanised 1A3B7

The ability of 1A3B7 to protect against a challenge dose of 100LD50 (approximately 30-50 pfu) VEEV strain TrD (subtype IA/B) was tested. Groups of Balb/c mice (7-9 weeks old, Charles River, U.K.) remained untreated or were injected intraperitoneally with 25, 50, 75 or 100 μg of antibody in 50-100 μl PBS. The challenge virus was administered subcutaneously 24 h later. After challenge, mice were observed twice daily for clinical signs of infection by an independent observer. Humane endpoints were used and these experiments therefore record the occurrence of severe disease rather than mortality. Even though it is rare for animals infected with virulent VEEV and showing signs of severe illness to survive, our use of humane endpoints should be considered when interpreting any virus dose expressed here as 50% lethal doses (LD50).

### Assessment of Cytokine Responses of Human Peripheral Blood Mononuclear Cells (PBMCs) Exposed to Murine and Humanised Versions of 1A3B-7.

PBMC Isolation:

Human blood (8 ml) from six individuals was collected in sodium citrate vacutainers (CPT citrate, Becton Dickenson, USA) in triplicate and processed within 2 hrs of collection. Tubes were centrifuged at 1500×g at room temperature for 25 mins (Sorvall RT6000). The plasma layer was removed and the PBMC layer washed in phosphate buffered saline (PBS, Gibco BRL, USA), followed by serum-free DMEM (Dulbecco's modified eagle medium; with Pen-Strep) via centrifugation at 400×g for 10 min (Jouan 3Ci). The PBMC pellet was resuspended in 1 ml serum free DMEM (with Pen-strep). Cells were counted using a haemocytometer.

PBMC Stimulation:

The PBMCs were cultured at 500,000 cells/well for 24 hrs in complete medium (RPMI 1640 (Invitrogen, Carlsbad, Calif.), 5% (v/v) Foetal calf serum (FCS), 100 U/ml penicillin and 100 μg/ml streptomycin, 1% L-glutamine, 0.1% MTG (Sigma-Aldrich, St Louis, Mo.) and then incubated undisturbed for a further 24 hours, in vitro, with either media alone (unstimulated, Blank), additional complete medium, 25 μg/well IgG from mouse serum, 25 μg/well IgG from human serum (reagent grade, ≧95% Sigma-Aldrich, St Louis, Mo.). 25 μg/well Mu 1 A3B-7, 25 μg/well Hu 1 A3B-7 or concanavalin A (Con A). Cell supernatants were assessed for cytokine content using a customized human flex cytometric bead array kit for IL-10, IL-12p70, IFN-γ. IL-6, IL-13, TNF-α and MCP-1 (BD Biosciences). Cytokine concentrations were measured via quantification of PE fluorescence of samples in reference to a standard curve generated by serial dilutions of control samples according to the manufacturer's instructions.

### Results:

Cloning of the Variable Domains of Murine 1A3B7 and In Vitro Assessment of scFv and Chimeric Murine/Human Antibody

The sequences of the variable light and variable heavy chain genes isolated from the hybridoma cell line 1A3B7 are shown in FIGS. 1A and B respectively. To confirm that the correct gene fragments had been extracted from the hybridoma cell line the V_{H} and V_{L} domains were linked together using a cellulase linker plus a human κ domain to form a scAb. The activity of this scAb molecule was evaluated in vitro against inactivated VEEV strain TC-83 (FIG. 2). This analysis showed that the scAb molecule comprised of the V_{H} and V_{L} domains harvested from the 1A3B7 hybridoma cell line detected immobilised VEEV antigen as expected and confirmed that the correct domains had been cloned.

The V_{H} and V_{L} domains from the murine antibody were used to provide a chimeric antibody molecule (murine variable regions, human IgG1 isotope constant regions). This molecule provided a positive control for use in further assays in comparison with the humanised forms of 1A3B7 due to the presence of the native variable regions, but allowed the use of the same detection reagents due to the presence of the human constant region of the antibody. This molecule was successfully produced in CHO DG44 cells and was found in in vitro activity assays to bind to inactivated TC83 in ELISA (FIG. 3). It is important to note in this instance that the binding curves associated with the murine parental and the murine chimeric do not overlap in this instance in response to dilution. This is due to the usage of two different detection antibodies in this assay (anti-mouse and anti-human) to reflect the different constant domains of each of the murine and chimeric molecules respectively.

### Selection of a Panel of Candidate Human Framework Scaffolds for CDR Grafting

The murine variable domains were subjected to a process of humanisation utilising the CDR grafting approach according to published methods (Jones P. T. et al., 1986, Nature, 321, 522-525). To identify human germline sequences most appropriate for supporting the murine CDR regions the anti VEEV 1A3B7 antibody variable domain sequences were aligned with the human V_{H} and V_{L} germ line sequences to reveal which human sequences were most similar or identical to the murine Vᵤ and V_{L} sequences. To mitigate the risks associated with the loss of antibody function as a result of the humanisation process, a panel of variant molecules were designed to provide 3 heavy chain and 3 light chain sequences for further evaluation.

Initial alignments indicated that for the heavy chain variable domain the most similar or identical human heavy chain germline sequences were DP-1 and DP-75. Furthermore, the analysis of the sequence of the murine antibody domains highlighted the presence of an unusual Isoleucine residue at position 94 (numbering based on Kabat E A et al., 1991) in the Framework 3 region of the murine heavy chain (highlighted in FIG. 4B). To take account of this characteristic of the murine antibody, this amino acid was retained in one of the versions of the humanised V_{H} gene. The version of humanised V_{H} 1A3B7 harbouring the unusual isoleucine residue is termed DP-75 CAI.

The three most similar light chain germline sequences were B1, A26 and L6. No unusual amino acids were identified in the light chain framework regions and these humanised genes were therefore constructed by conventional CDR grafting with no other amendments to the human frameworks. Of note however, is that the 1A3B7 murine V_{L} domain possesses an unusually long CDR1 domain (15 amino acids). The B1 germline sequence is also unusual in that it naturally supports a CDR1 sequence of the same size and therefore has an additional advantageous characteristic for the humanisation process further to overall sequence similarity or identity.

The alignments of the V_{H} and V_{L} chain sequences after the grafting of the murine CDR regions is provided in FIGS. 4 A and B respectively.

The nine permutations of the variable domain variants were constructed by using overlapping oligonucleotides in overlap extension PCR. The resultant amplicons were cloned into T vector (Promega) and their sequences determined.

### Production of Humanised Recombinant 1A3B7 in Mammalian Cell Culture

All nine possible combinations of V_{L} and V_{H} were cloned into Haptogen's antibody expression vector (pHEE) A DNA sample from each of these nine constructs was sent for sequencing and determined to be correct. The expression vectors made for this work were as follows:
pHEE1A3B7 VEEV DP1 VH IgG1 A26 Kappa
pHEE1A3B7 VEEV DP1 VH IgG1 L6 Kappa
pHEE1A3B7 VEEV DP1 VH IgG1 B1 Kappa
pHEE1A3B7 VEEV DP75 VH IgG1 A26 Kappa
pHEE1A3B7 VEEV DP75 VH IgG1 L6 Kappa
pHEE1A3B7 VEEV DP75 VH IgG1 B1 Kappa
pHEE1A3B7 VEEV DP75 VH CAI IgG1 A26 Kappa
pHEE1A3B7 VEEV DP75 VH CAI IgG1 L6 Kappa
pHEE1A3B7 VEEV DP75 VH CAI IgG1 B1 Kappa

Each of the panel of nine variants was expressed in low levels in mammalian cell culture. The concentration of the secreted 1A3B7 antibody variants was determined by capture ELISA. No expression could be observed for any of the constructs utilising the DP 1 heavy chain variant. Further work with these constructs was therefore halted. The six constructs that directed the production of antibody were grown further and antibody samples were used in ELISA to determine binding to inactivated TC83 VEEV in ELISA. Samples of chimeric 1A3B7 antibody and an irrelevant human IgG1/kappa antibody were used as positive and negative controls to assess the binding of the transiently expressed humanised 1A3B7 antibodies to immobilized VEEV coated onto ELISA plates (FIG. 5). These results illustrate that binding of the VEEV DP75 VH IgG1 A26 Kappa, VEEV DP75 VH IgG1 L6 Kappa, VEEV DP75 VH IgG1 B1 Kappa, VEEV DP75 VH CAI IgG1 A26 Kappa, VEEV DP75 VH CAI IgG1 L6 Kappa variants is not detectable with only the VEEV DP75 VH CAI IgG1 B1 Kappa variant giving any signal in the binding ELISA. The results show that only one combination of the humanised heavy and light 1A3B7 variable regions results in an antibody that bind to VEEV antigen in the ELISA binding assay (FIG. 5). Both the chimaeric and humanized 1A3B7 VEEV DP75 VH CAI/IgG1 B1 Kappa antibodies bind to immobilized VEEV antigen in a similar manner (within 2 fold). This discrepancy in binding could be accounted for by experimental error when diluting antibody samples and calculating antibody concentration by ELISA

### Activity of Humanised 1A3B7 in ELISA and Neutralisation Assays

In order to ensure that the range of VEEV reactivity had been retained during the humanisation process, the antibody was tested in comparison to the murine 1A3 B7 in an ELISA using antigens from multiple strains (FIG. 7). Comparable levels of reactivity for both the murine and humanised versions of 1A3B7 were observed for all strains, with the exception of 75V (subtype IF) and Pixuna subtype IV). A more detailed analysis of the binding characteristics of the humanised antibody was then undertaken using a dilution series of antibody to assess the relative binding to the positive strains of VEEV (FIG. 8). These two assays indicated that the breadth of specificity of the antibody had in been retained. The ability of the humanised 1A3B7 to neutralise virus was also assessed in in vitro cell culture against three representative strains of VEEV. This analysis showed that the virus had retained a comparable ability to neutralise VEEV from subtypes IA/B (strain TrD), II (strain Fe37c) or III (strain BeAn8) at a comparable level of that of the original antibody (FIG. 9). To provide confidence that the humanised molecule no longer retained murine epitopes, the reactivity of the humanised molecule to a polyclonal anti-mouse antibody was evaluated in comparison to a further murine anti-VEEV antibody 1A4A1 (FIG. 10A). This analysis indicated that the protein was no longer detected by the anti-mouse antibody. In comparison the humanised molecule reacted well to an anti-human polyclonal antibody in a comparable assay using the same controls (FIG. 10B).

Activity of humanized 1A3B7 in protecting mice from lethal VEEV challenge The humanised 1A3B7 antibody was assessed for its ability to provide protection against lethal challenge in a small animal model of disease. Balb/c mice were pre-treated with a range of antibody doses. 24 hours later, the animals were challenged with 100LD₅₀ of VEEV (strain IA/B) and monitored for 14 days. The results (Table 1) show that the humanised antibody generates significantly higher levels of protection than the original murine molecule (chi sq 6.6; critical score 3.841, p<0.05) (Table I).

### Table 1:

Survival of Balb/c mice pre-treated with antibody before challenge with 100LD₅₀ of VEEV. Figures show number of surviving mice/total number of mice challenged and percent survival in parentheses.

TABLE 1

| Antibody dose | 1A3B7 | h1A3B7 |
|---|---|---|
| 25 μg | 4/5 (80%) | 10/10 (100%) |
| 50 μg | 5/5 (100%) | 10/10 (100%) |
| 75 μg | 3/5 (60%) | 10/10 (100%) |
| 100 μg | 4/5 (80%) | 10/10 (100%) |

### Comparison of the Immunostimulatory Properties of Hu1A3B-7 and Mu1A3B-7 In Vitro Using Human PBMCs

The biological properties of Hu1A3B-7 were further investigated using an in vitro cytokine secretion assay. PBMCs from human donors were incubated in the presence of either Hu1A3B-7 or Mu1A3B-7 for 24 h. The release of inflammatory cytokines was then monitored. Experiments were performed at least twice using control human and murine antibodies for comparison and a positive control of ConA. Data shown are representative of these experiments (FIG. 11). Stimulation of human PBMCs with Mu1A3B-7 and a control murine antibody resulted in secretion of significantly higher levels of cytokines, MCP-1, IL-6, TNF α, and IL-10 compared to PBMCs stimulated with Hu1A3B-7 and a fully human control antibody (FIG. 11). A similar pattern was seen when comparing the cytokine response of human PBMCs stimulated with murine or human IgG controls. Levels of IL-12p70, INF-γ, and IL-13 were also analysed but were found to be at the lower limit of detection, however ConA still elicited a positive response for all these cytokines (data not shown). This suggests that Hu1A3B-7 may appear more “human-like” to the immune system and has less potential to non-specifically stimulate an inflammatory cytokine response than the parental murine antibody.

### LIST OF FIGURES

FIG. 1: annotated sequence from murine antibody 1A3B7 A) Variable light chain, B) Variable heavy chain.

FIG. 2: Evaluation of the retention of the antigen binding activity of the putative V_{H} and V_{L} domains isolated from the hybridoma cell line 1A3B7 in scAb format. The activity of the scAb was compared to the activity of the parental murine antibody using non-specific scAb and murine monoclonal as negative controls. The activity of the variable domains when displayed on the surface of M13 filamentous phage is also shown.

FIG. 3: Evaluation of the relative antigen binding activity of Chimeric 1A3B7 (murine V_{H} and V_{L} grafted onto human IgG1 isotype contact regions) in comparison to the murine parental molecule.

FIG. 4: Alignment of humanised sequences generated for A) the V_{L} domain of antibody 1A3B7 and B) the V_{H} domain of 1A3B7, The amino acid sequences of the humanised variants are shown in comparison with the murine parental molecule for each variable domain. The CDR regions grafted on to each framework region are shown highlighted in grey. Amino acid residues that have changed from the original murine molecule to reflect the sequences of the human germline are shown boxed. The unusual isoleucine found within the murine VH domain of 1A3B7 and retained in one of the humanised VH variants (DP75 (CAI)), is shown highlighted by cross hatching.

FIG. 5: Comparison of the binding profiles of humanised 1A3B7 antibody molecule V_{H} DP75(CAI)/V_{L} B1 in comparison with the parent murine 1A3B7 and the chimeric 1A3B7 (murine variable domains with human constant backbone). Binding profiles of the murine molecule with the humanised and chimeric molecules are not comparable across the dilution series due the necessity to use different anti-species detection regents within this ELISA.

FIG. 6: Analysis of purified hu1A3B7 (DP75 CAI/BI) by denaturing SDS-PAGE. Lane 1 is loaded with 1 μg of a non-specific human IgG1 molecule that was electrophoresed as a positive control. Lane 2 is loaded with 1 μg of DP75 CAI/BI. The denaturing SDS-Page was stained using GelCode™ stain to show electrophoresis of the heavy and light chains of the recombinant molecule.

FIG. 7: Comparison of the relative binding efficiency of Hu1A3B7 to a range of VEEV strains in comparison to the parental murine 1A3B7 antibody. Each antibody (10 μg/ml) was tested by ELISA using antigen prepared from VEEV strains TC-83, TrD, P676, 3880, Mena II, 78V, Fe37c, BeAn8, Pixuna, CaAr508 and AG80 (subtypes IA/B, IA/B, IC, ID, IE, IF, II, IIIA, IV, V and VI respectively). Negative control antigen was prepared from cells that had been mock infected. n=6 for all data points, 95% confidence intervals are shown.

FIG. 8: Comparison of the relative neutralisation activity of Hu1A3B7 to the parental murine 1A3B7 antibody. Incubation of virus with media was used as a positive control for virus infectivity in cell culture. A reduction in titre as compared to control wells without MAB, of equal to or greater than 3-fold (0.48 log 10) or the production of obviously smaller “pinpoint” plaques compared to the plaque size in controls was considered indicative of neutralisation. 95% Confidence limits are shown.

FIG. 9: ELISA analysis of the binding of Hu1 A3B7 to a range of VEEV strains over a dilution series of antibody

FIG. 10: Analysis of the reactivity of Hu1A3B7 and Mu1A3B7 to A) polyclonal anti-mouse and B) anti-human detection antibodies.

FIG. 11: Secretion of inflammatory cytokines from human Peripheral Blood Mononuclear Cells (PBMCs) stimulated for 24 h with murine, human and humanised antibodies. Levels of IL-6, TNF-α, IL-10 and MCP-1 were measured by CBA; all cytokine data are expressed as μg/ml.=significant difference between the Mu1A3B-7 and Hu1 A3B-7 stimulated human PBMCs (Mann Whitney U test, *=p<0.05 and **=p<0.01). Experiments were performed at least twice and data shown are a representative experiment. Values represent the mean±SE for 6 samples per group.

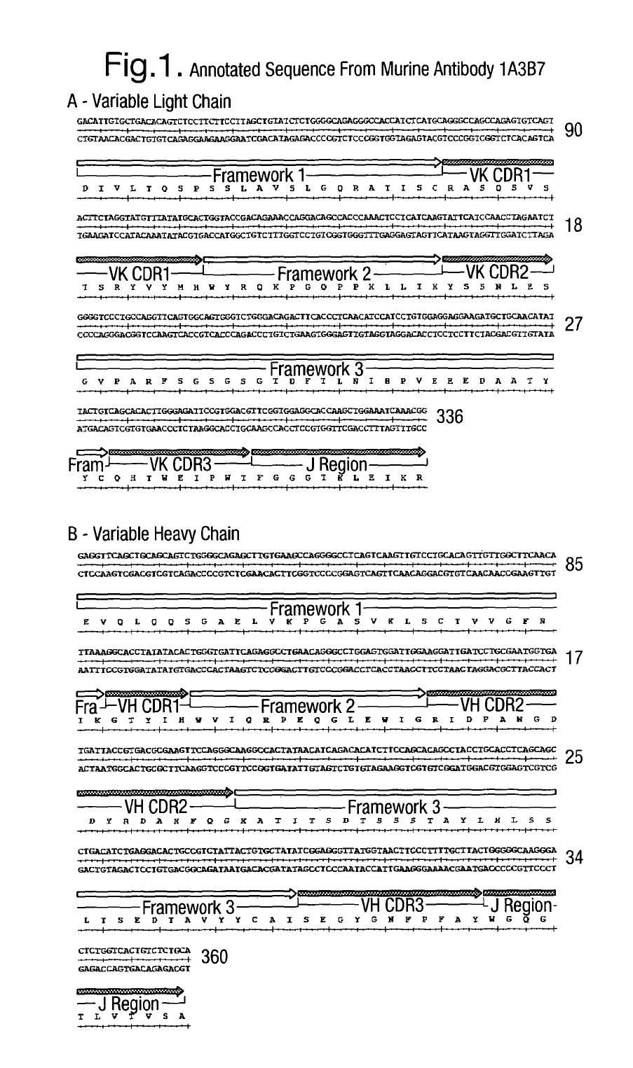

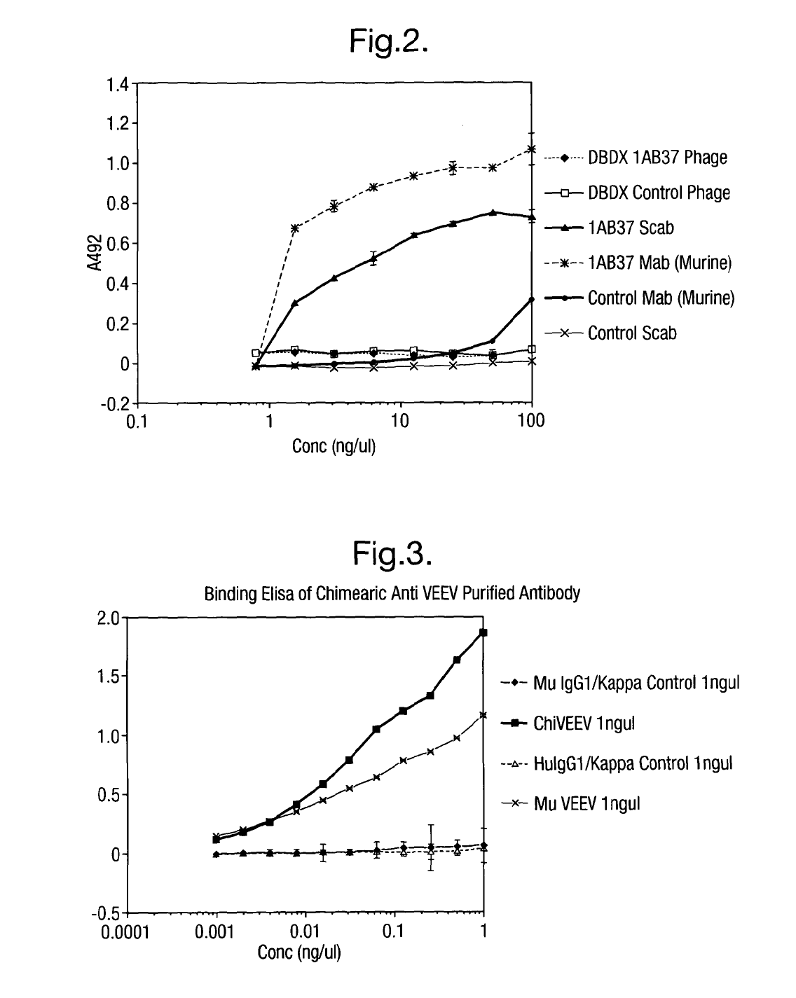

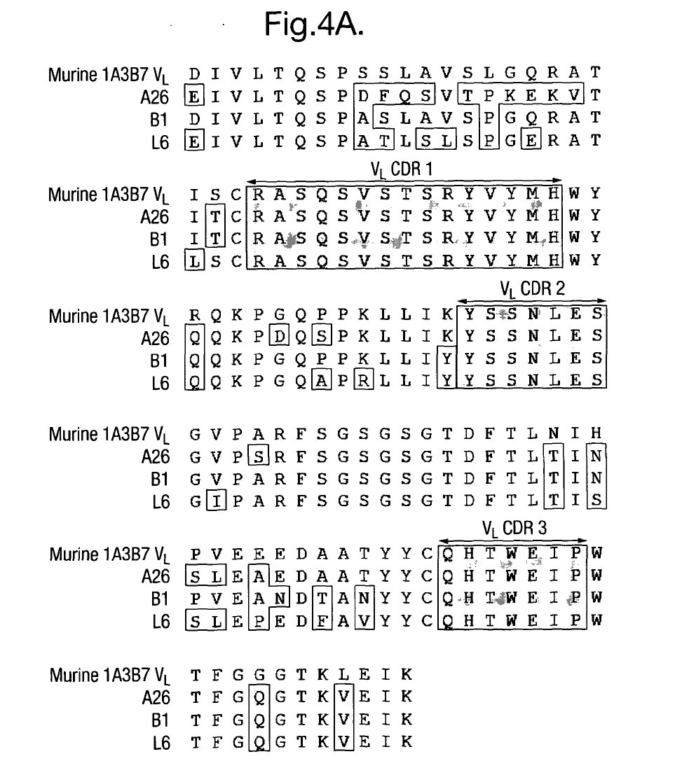

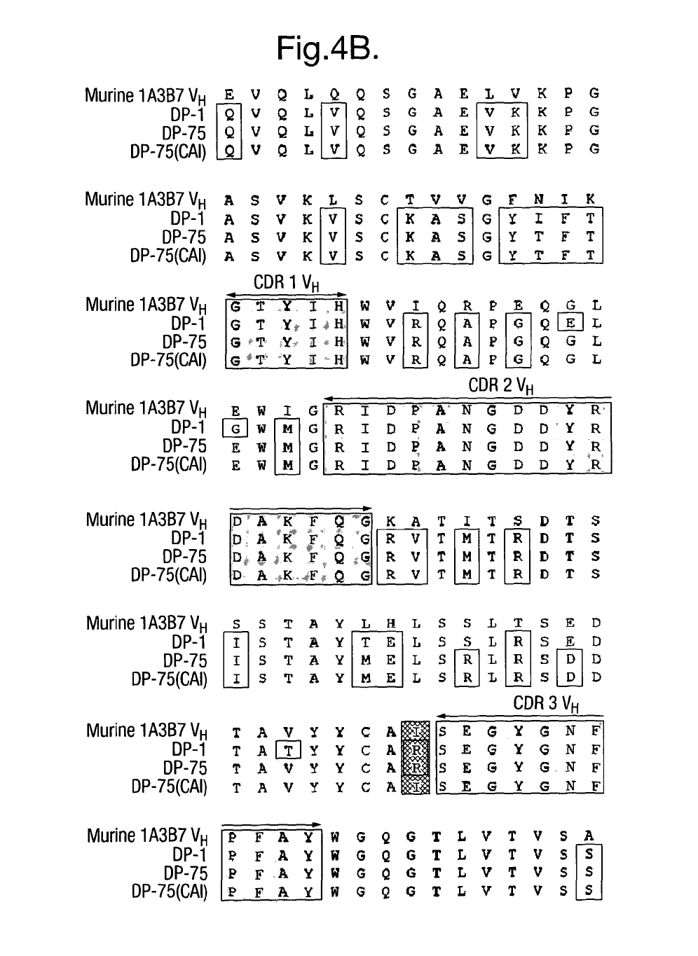

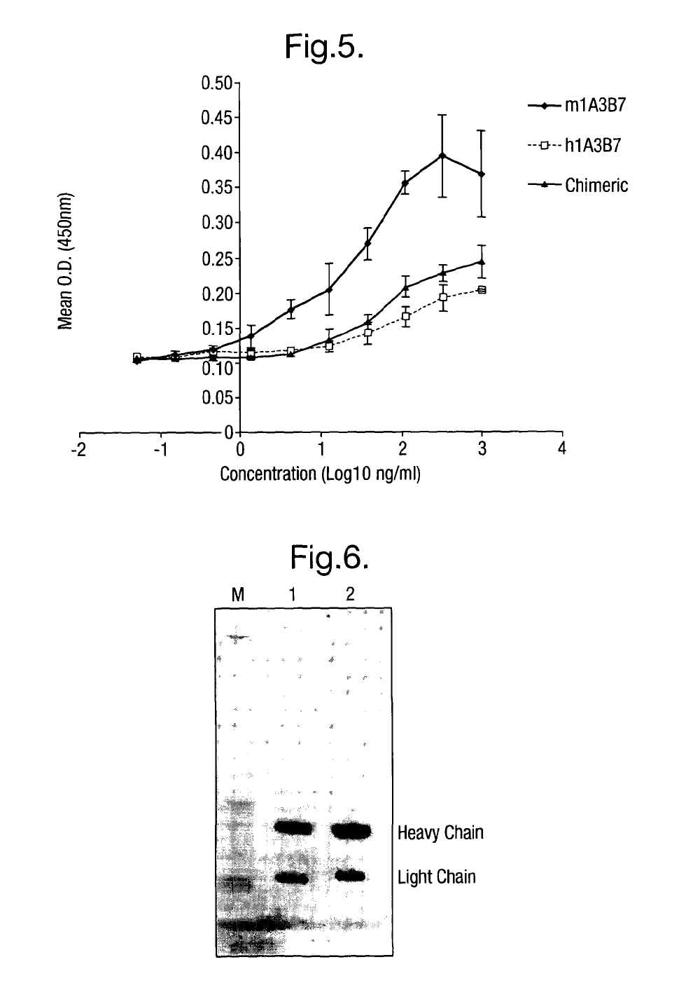

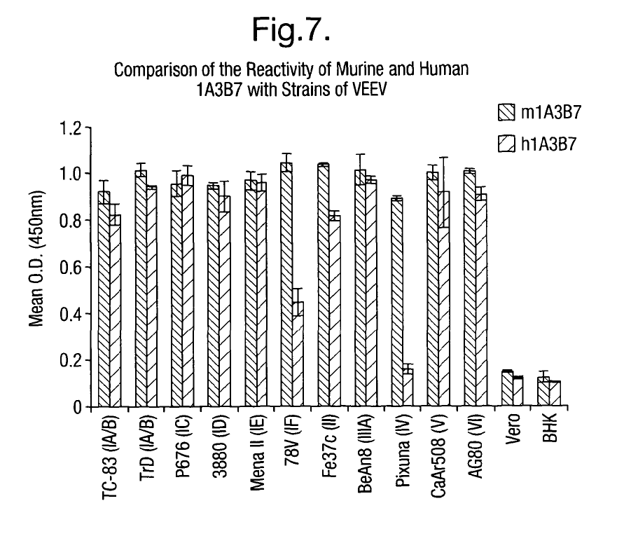

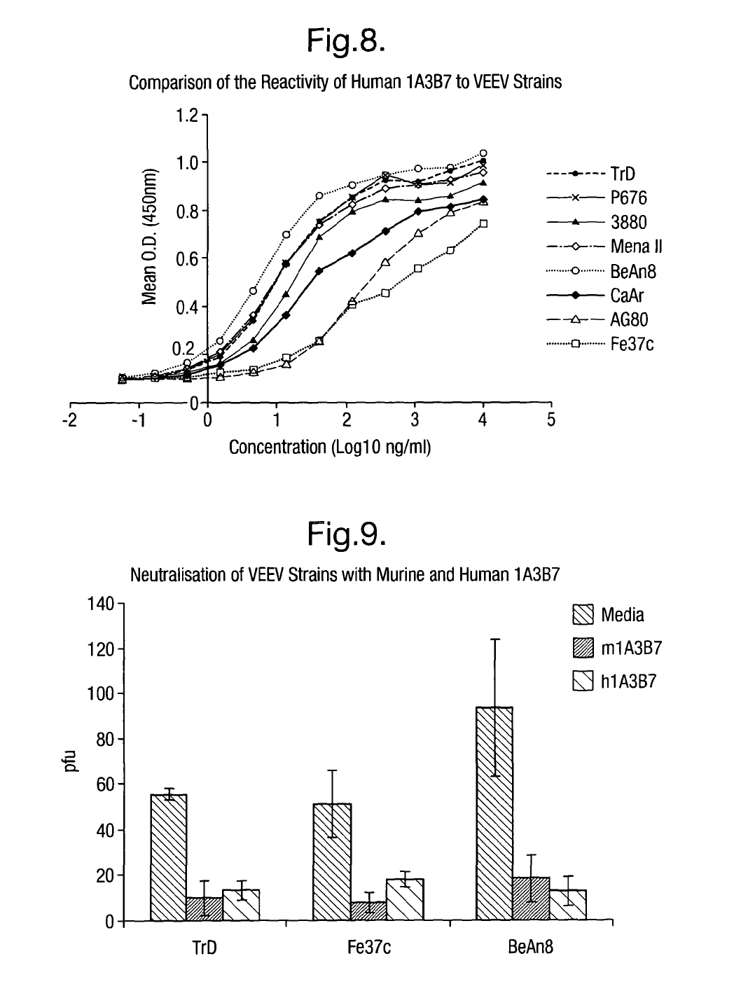

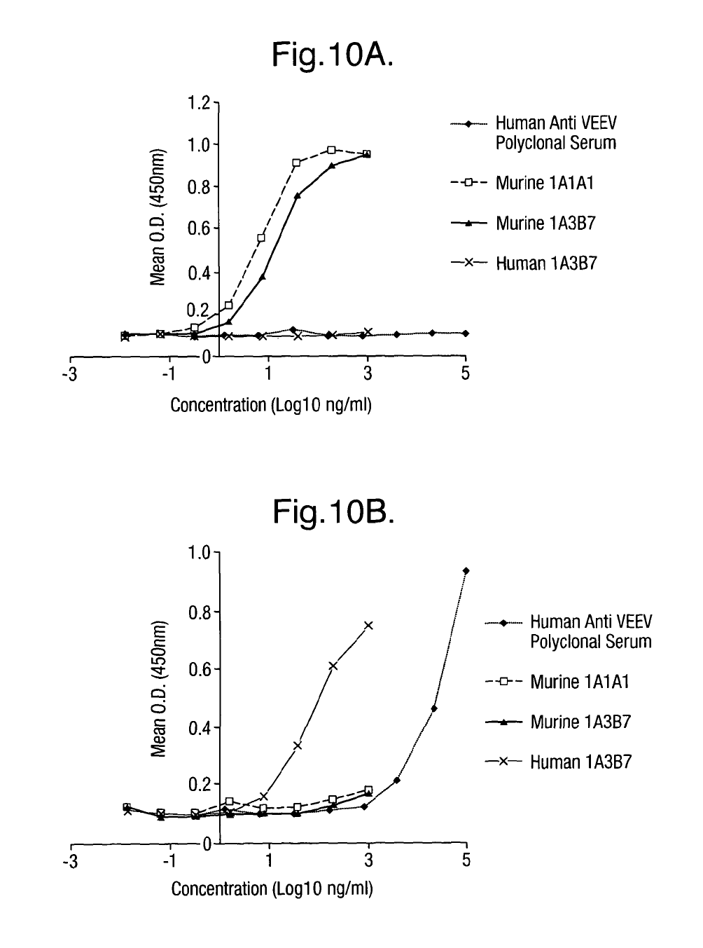

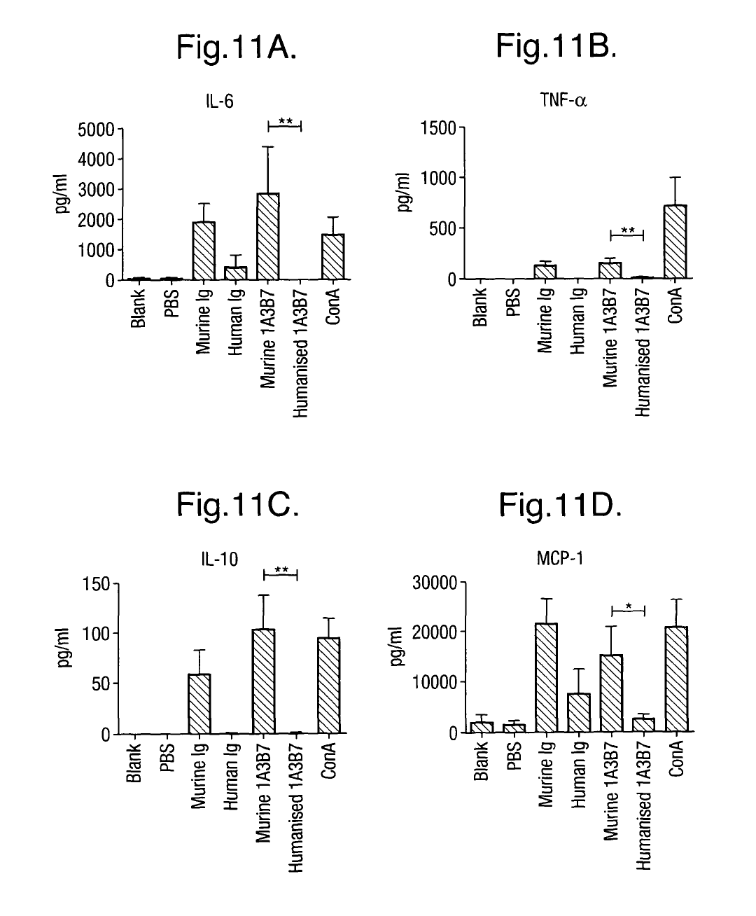

This work describes the successful humanisation of a broadly reactive murine anti-VEEV antibody through the use of a CDR grafting approach (Jones P. T. et al., 1989, Nature, 321, 522-525). An evaluation of a panel of nine antibody variants was performed leading to isolation of one candidate molecule that retained the breadth of activity, affinity and neutralisation activity of the original parent antibody in in vitro assays. Use of the antibody in in vivo passive protection studies comparable to previous work (Phillpotts R., 2006, Virus Research, 120, 107-112) has shown that this antibody also retains the protective qualities of the parent antibody to lethal challenge with VEEV in mice.

Full amino acid sequence of murine anti-VEEV monoclonal antibody 1A3B7. The variable domains of each chain of the antibody are shown underlined, and the mouse constant light (kappa) and constant heavy (IgG2 isotype) for each chain are shown without underline. The bold type in SEQ ID No: 1 and SEQ ID No:2 illustrates the CDRs within the variable chains. The CDRs are also listed separately as SEQ ID 3-8. The isotype of the 1A3B7 antibody was identified through isotype testing of the original cell line.

Seq ID No 1: Murine variable light chain
DIVLTQSPSSLAVSLGQRATISCRASQSVSTSRYVYMHWYRQKPGQPPKLLIKYSSNLESGV
PARFSGSGSGTDFTLNIHPVEEEDAATYYCQHTWEIPWTFGGGTKLEIKRRADAAPTVSIFP
PSSEQLTSGGASVVCFLNNEYPKDINVKWKIDGSERQNGVLNSWTDQDSKDSTYSMSSTLTL
TKDEYERHNSYTCEATHKTSTSPIVKSFNRNEC
Seq ID No 2: Murine variable heavy chain
EVQLQQSGAELVKPGASVKLSCTVVGFNIKGTYIHWVIQRPEQGLEWIGRIDPANGDDYRDA
KFQGKATITSDTSSSTAYLHLSSLTSEDTAVYYCAISEGYGNFPFAYWGQGTLVTVSAAKTT
APSVYPLAPVCGDTTGSSVTLGCLVKGYFPEPVTLTWNSGSLSSGVHTFPAVLQSDLYTLSS
SVTVTSSTWPSQSITCNVAHPASSTKVDKKIEPRGPTIKPCPPCKCPAPNLLGGPSVFIFPP
KIKDVLMISLSPIVTCVVVDVSEDDPDVQISWFVNNVEVHTAQTQTHREDYNSTLRVVSALP
IQHQDWMSGKEFKCKVNNKDLPAPIERTISKPKSVRAPQVYVLPPPEEEMTKKQVTLTCMVT
DFMPEDIYVEWTNNGKTELNYKNTEPVLDSDGSYFMYSKLRVEKKNWVERNSYSCSVVHEGL
HNHHTTKSFSRTPGK
CDR 1 (H1) Seq ID No 3:
GTYIH
CDR2 (H2) Seq ID No 4:
RIDPANGDDYRDAKFQG
CDR3 (H3) Seq ID No 5:
SEGYGNFPFAY
CDR4 (L1) Seq ID No 6:
RASQSVSTSRYVYMH
CDR5 (L2) Seq ID No 7:
YSSNLES
CDR6 (L3) Seq ID No 8:
QHTWEIP
Human light chain variable framework B1 Seq ID No 9:
IGSGAPLLWILLLWAPSCNGDIVLTQSPASLAVSPGQRATITCRASESVSFLGINLI
HWYQQKPGQPPKLLIYQASNKDTGVPARFSGSGSGTDFTLTINPVEANDTANYYCLQ
SKNFP
Human heavy chain variable framework DP75
Seq ID No 10
QVQLVQSGAEVKKPGASVKVSCKASGYTFTGYYMHWVRQAPGQGLEWMGWINPNSGG
TNYAQKFQGRVTMTRDTSISTAYMELSRLRSDDTAVYYCAR
Humanised heavy chain
Seq ID No: 11
QVQLVQSGAEVKKPGASVKVSCKASGYTFTGTYIHWVRQAPGQGLEWMGRIDPANGDDYRDA
KFQGRVTMTRDTSISTAYMELSRLRSDDTAVYYCAISEGYGNFPFAYWGQGTLVTVSS
Humanised light chain
Seq ID No: 12
DIVLTQSPASLAVSPGQRATITCRASQSVSTSRYVYMHWYQQKPGQPPKLLIYYSSNLESGV
PARFSGSGSGTDFTLTINPVEANDTANYYCQHTWEIPWTFGQGTKVEIK

1. An anti-VEEV humanised antibody or a fragment thereof comprising a framework of 1, 2, 3, 4, 5 or 6 CDR regions independently selected from SEQ ID Nos: 3, 4, 5, 6, 7 or 8, wherein the antibody or fragment comprises in the framework at least one amino acid that positively influences the binding and/or activity of the antibody from the original murine antibody IA3B7.

2. The anti-VEEV antibody according to claim 1, wherein the antibody or fragment comprises at least the CDR sequence of SEQ ID No: 5 and an isoleucine amino acid corresponding to isoleucine H94 in the original murine antibody 1A3B7.

3. The anti-VEEV antibody according to claim 1, wherein antibody has the sequence shown in SEQ ID No: 12, or a sequence 90% homologous thereto.

4. The anti-VEEV antibody according to claim 1, wherein the antibody or fragment comprises the heavy chain variable region sequence of SEQ ID No: 11 or a sequence 90% homologous thereto.

5. The anti-VEEV antibody or fragment thereof according to claim 1, wherein the fragment is an Fab′ fragment.

6. A pharmaceutical composition comprising an antibody or fragment as defined in claim 1, and a pharmaceutically acceptable excipient.

7. The pharmaceutical composition of claim 6, wherein the composition is for infusion.

8. A method for the treatment of VEEV comprising administering to an individual a pharmaceutical composition comprising an anti-VEEV humanised antibody or a fragment thereof comprising a framework 1, 2, 3, 4, 5 or 6 CDR regions independently selected from SEQ ID Nos: 3, 4, 5, 6, 7 or 8, wherein the antibody or fragment comprises in the framework at least one amino acid that positively influences the binding and/or activity of the antibody from the original murine antibody IA3B7.

9. The method of claim 8 wherein the pharmaceutical composition further comprises a pharmaceutically acceptable excipient.

10. The method of claim 8 wherein the pharmaceutical composition is administered by infusion.

11. The method of claim 9 wherein the pharmaceutical composition is administered by infusion.


SEQUENCE LISTING (25 sequences)

SEQ ID NO: 1 - protein, 219 aa, Mus musculus
DIVLTQSPSSLAVSLGQRATISCRASQSVSTSRYVYMHWYRQKPGQPPKLLIKYSSNLESGVPARFSGSGSGTDFTLNIHPVEEEDAATYYCQHTWEIPWTFGGGTKLEIKRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 449 aa, Mus musculus
EVQLQQSGAELVKPGASVKLSCTVVGFNIKGTYIHWVIQRPEQGLEWIGRIDPANGDDYRDAKFQGKATITSDTSSSTAYLHLSSLTSEDTAVYYCAISEGYGNFPFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 5 aa, Mus musculus
GTYIH

SEQ ID NO: 4 - protein, 17 aa, Mus musculus
RIDPANGDDYRDAKFQG

SEQ ID NO: 5 - protein, 11 aa, Mus musculus
SEGYGNFPFAY

SEQ ID NO: 6 - protein, 15 aa, Mus musculus
RASQSVSTSRYVYMH

SEQ ID NO: 7 - protein, 7 aa, Mus musculus
YSSNLES

SEQ ID NO: 8 - protein, 7 aa, Mus musculus
QHTWEIP

SEQ ID NO: 9 - protein, 119 aa, Homo sapiens
IGSGAPLLWILLLWAPSCNGDIVLTQSPASLAVSPGQRATITCRASESVSFLGINLIHWYQQKPGQPPKLLIYQASNKDTGVPARFSGSGSGTDFTLTINPVEANDTANYYCLQSKNFP

SEQ ID NO: 10 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 11 - protein, 120 aa, Artificial sequence
  Synthetic sequence Humanised heavy chain
QVQLVQSGAEVKKPGASVKVSCKASGYTFTGTYIHWVRQAPGQGLEWMGRIDPANGDDYRDAKFQGRVTMTRDTSISTAYMELSRLRSDDTAVYYCAISEGYGNFPFAYWGQGTLVTVSS

SEQ ID NO: 12 - protein, 111 aa, Artificial sequence
  Synthetic sequence Humanised light chain
DIVLTQSPASLAVSPGQRATITCRASQSVSTSRYVYMHWYQQKPGQPPKLLIYYSSNLESGVPARFSGSGSGTDFTLTINPVEANDTANYYCQHTWEIPWTFGQGTKVEIK

SEQ ID NO: 13 - DNA, 336 bp, Mus musculus
  CDS (1)..(336)
gacattgtgctgacacagtctccttcttccttagctgtatctctggggcagagggccaccatctcatgcagggccagccagagtgtcagtacttctaggtatgtttatatgcactggtaccgacagaaaccaggacagccacccaaactcctcatcaagtattcatccaacctagaatctggggtccctgccaggttcagtggcagtgggtctgggacagacttcaccctcaacatccatcctgtggaggaggaagatgctgcaacatattactgtcagcacacttgggagattccgtggacgttcggtggaggcaccaagctggaaatcaaacgg

SEQ ID NO: 14 - protein, 112 aa, Mus musculus
DIVLTQSPSSLAVSLGQRATISCRASQSVSTSRYVYMHWYRQKPGQPPKLLIKYSSNLESGVPARFSGSGSGTDFTLNIHPVEEEDAATYYCQHTWEIPWTFGGGTKLEIKR

SEQ ID NO: 15 - DNA, 336 bp, Mus musculus
ccgtttgatttccagcttggtgcctccaccgaacgtccacggaatctcccaagtgtgctgacagtaatatgttgcagcatcttcctcctccacaggatggatgttgagggtgaagtctgtcccagacccactgccactgaacctggcagggaccccagattctaggttggatgaatacttgatgaggagtttgggtggctgtcctggtttctgtcggtaccagtgcatataaacatacctagaagtactgacactctggctggccctgcatgagatggtggccctctgccccagagatacagctaaggaagaaggagactgtgtcagcacaatgtc

SEQ ID NO: 16 - DNA, 360 bp, Mus musculus
  CDS (1)..(360)
gaggttcagctgcagcagtctggggcagagcttgtgaagccaggggcctcagtcaagttgtcctgcacagttgttggcttcaacattaaaggcacctatatacactgggtgattcagaggcctgaacagggcctggagtggattggaaggattgatcctgcgaatggtgatgattaccgtgacgcgaagttccagggcaaggccactataacatcagacacatcttccagcacagcctacctgcacctcagcagcctgacatctgaggacactgccgtctattactgtgctatatcggagggttatggtaacttcccttttgcttactgggggcaagggactctggtcactgtctctgca

SEQ ID NO: 17 - protein, 120 aa, Mus musculus
EVQLQQSGAELVKPGASVKLSCTVVGFNIKGTYIHWVIQRPEQGLEWIGRIDPANGDDYRDAKFQGKATITSDTSSSTAYLHLSSLTSEDTAVYYCAISEGYGNFPFAYWGQGTLVTVSA

SEQ ID NO: 18 - DNA, 360 bp, Mus musculus
tgcagagacagtgaccagagtcccttgcccccagtaagcaaaagggaagttaccataaccctccgatatagcacagtaatagacggcagtgtcctcagatgtcaggctgctgaggtgcaggtaggctgtgctggaagatgtgtctgatgttatagtggccttgccctggaacttcgcgtcacggtaatcatcaccattcgcaggatcaatccttccaatccactccaggccctgttcaggcctctgaatcacccagtgtatataggtgcctttaatgttgaagccaacaactgtgcaggacaacttgactgaggcccctggcttcacaagctctgccccagactgctgcagctgaacctc

SEQ ID NO: 19 - protein, 111 aa, Mus musculus
DIVLTQSPSSLAVSLGQRATISCRASQSVSTSRYVYMHWYRQKPGQPPKLLIKYSSNLESGVPARFSGSGSGTDFTLNIHPVEEEDAATYYCQHTWEIPWTFGGGTKLEIK

SEQ ID NO: 20 - protein, 111 aa, Mus musculus
EIVLTQSPDFQSVTPKEKVTITCRASQSVSTSRYVYMHWYQQKPDQSPKLLIKYSSNLESGVPSRFSGSGSGTDFTLTINSLEAEDAATYYCQHTWEIPWTFGQGTKVEIK

SEQ ID NO: 21 - protein, 111 aa, Mus musculus; identical to SEQ ID NO: 12

SEQ ID NO: 22 - protein, 111 aa, Mus musculus
EIVLTQSPATLSLSPGERATLSCRASQSVSTSRYVYMHWYQQKPGQAPRLLIYYSSNLESGIPARFSGSGSGTDFTLTISSLEPEDFAVYYCQHTWEIPWTFGQGTKVEIK

SEQ ID NO: 23 - protein, 120 aa, Mus musculus
QVQLVQSGAEVKKPGASVKVSCKASGYIFTGTYIHWVRQAPGQELGWMGRIDPANGDDYRDAKFQGRVTMTRDTSISTAYTELSSLRSEDTATYYCARSEGYGNFPFAYWGQGTLVTVSS

SEQ ID NO: 24 - protein, 120 aa, Mus musculus
QVQLVQSGAEVKKPGASVKVSCKASGYTFTGTYIHWVRQAPGQGLEWMGRIDPANGDDYRDAKFQGRVTMTRDTSISTAYMELSRLRSDDTAVYYCARSEGYGNFPFAYWGQGTLVTVSS

SEQ ID NO: 25 - protein, 120 aa, Mus musculus; identical to SEQ ID NO: 11